(19) United States
(12) Patent Application Publication
(10) Pub. No.: US 2026/0259190 A1
(43) Pub. Date: Sep. 3, 2026

(54) DEVICE AND METHOD FOR AUTOMATED CONFIGURATION OF SOFT CLAY FOUNDATION BASED ON MODEL TANK TEST

(71) Applicant: SOUTHEAST UNIVERSITY, Jiangsu (CN)

(72) Inventors: Yan ZHUANG, Jiangsu (CN); Jinxin LI, Jiangsu (CN); Shunlei HU, Jiangsu (CN); Yunsheng LU, Jiangsu (CN); Hu FAN, Jiangsu (CN); Xiaoyan CUI, Jiangsu (CN)

(73) Assignee: SOUTHEAST UNIVERSITY, Jiangsu (CN)

(21) Appl. No.: 18/878,654

(22) PCT Filed: Feb. 27, 2024
(86) PCT No.: PCT/CN2024/078747

§ 371 Date: Dec. 24, 2024

(30) Foreign Application Priority Data
May 12, 2023 (CN) 202310534122.8

(51) Int. Cl. G01N 33/42

(52) U.S. Cl. CPC G01N 33/42

(57) ABSTRACT

A device for automated configuration of a soft clay foundation based on a model tank test including a model tank device, a clay crushing device, a clay mixing device, a coarse-fine aggregate elevating device and a clay filling device. A model tank has a gravel layer and a sandy clay supporting layer. The clay crushing device includes a clay crushing chamber and a motor, and the motor drives, by means of a belt, blades inside the clay crushing chamber to rotate. The clay mixing device is connected to an outlet of the clay crushing device to convey a finished product. A coarse-fine aggregate elevating device has cuboidal blocks, which are connected to the motor by means of a chain and a gear to deliver aggregates into a mixing barrel. A scissor-type elevating device is installed inside the rail to push the finished product into the model tank.

### TECHNICAL FIELD

The invention relates to the technical field of soft clay foundation for transportation ground, and in particular to a device and method for automated configuration of a soft clay foundation based on a model tank test.

### TECHNICAL BACKGROUND

China, as a traditional infrastructure power, has made remarkable achievements in transportation construction over a period of time, and its construction of highways and high-speed railways is in full swing, which greatly increases the communication and contacts across various regions of China. However, on the other hand, there are many problems emerging in engineering construction. Due to the vast land area of China, the transportation construction is confronted with extremely complex engineering geological conditions, for example, the collapsible loess in the northwest region and the seasonally frozen ground in Qinghai-Tibet Plateau. These special soil foundations have brought great challenges to the transportation construction in China. Soft clay is widely distributed in China, and there is a certain amount of sedimentary distribution of soft clay foundations from the southeast coastal regions, to the inland river and lake marshy regions. This soft clay has such physical and mechanical properties as high water content, poor bearing capacity, and high compressibility, which are adverse to the construction and long-term operation of transportation facilities. In front of these soil masses adverse to the engineering, a series of foundation treatment measures, such as road embankment filling with light fillers, vacuum consolidation or the like, have been developed in the field of civil engineering in its long course of history. Reinforced pile-bearing type road embankment is a classic method for dealing with soft clay foundations, in which piles are placed in a soft clay foundation, road embankments and reinforcements are arranged on the soft clay foundation, and most of the loads passing through the upper road embankments are transferred to the piles due to the rigid difference between the piles and the soil. In the reinforced pile-bearing type road embankment, there are two main load transfer effects, namely, a soil arch effect and a tensioned membrane effect. With these two effects, the upper loads can be effectively transferred to the piles and eventually to a lower supporting layer.

In the reinforced pile-bearing type road embankment, the tensioned membrane effect is a very important load transfer mechanism, of which the exertion degree is affected by the upper load form and the road embankment height, in particular by the property of a reinforced bedding. Regarding this, some studies have been carried out before, which however, are mostly limited to theoretical studies that lack experimental research results on the tensioned membrane effect. Therefore, there is an urgent need for a test device and method for studying the influence of factors such as the form of dynamic load and related parameters, the height of the upper road embankment, and the number of geogrid layers on the degree of exertion of the tensioned membrane effect in the reinforced pile-bearing road embankment.

### SUMMARY OF THE INVENTION

In view of the defects in the prior art, the technical problem to be solved by the invention is to provide a device for automated configuration of a soft clay foundation based on a model tank test to improve the working efficiency of the procedure of soft clay preparation and filling in the model tank test, allowing for normalized and automated operation of the model tank test, thereby exploring the influence of different factors on the tensioned membrane effect in the reinforced pile-bearing type road embankment.

To solve the technical problem mentioned above, the invention employs a technical solution as follows: a device for automated configuration of a soft clay foundation based on a model tank test includes: a model tank device, a clay crushing device, a clay mixing device, a coarse-fine aggregate elevating device, and a clay filling device.

The model tank device includes a model tank, a gravel layer, a sandy clay supporting layer, rigid end-bearing piles, an actuator, a first water supply pipeline, and a top cross beam.

The model tank is a square tank with an opening in the top, and the gravel layer is laid on the bottom of the model tank, the sandy clay supporting layer is laid above the gravel layer, and a plurality of rigid end-bearing piles is embedded in the sandy clay supporting layer; and the top cross beam is fixed to the top of the model tank, and the actuator is suspended on the top cross beam.

The clay crushing device includes a feed port guard plate, a clay crushing chamber, a helical blade set, a first motor, a discharge guide slot, a first cover plate, and a belt.

The feed port guard plate is obliquely and fixedly connected to the clay crushing chamber, the helical blade set is disposed inside the clay crushing chamber, a lower part of the clay crushing chamber is connected to the discharge guide slot, and the first motor drives, by means of the belt, the helical blade set to rotate; the first cover plate is located at an outlet of the discharge guide slot.

The coarse-fine aggregate elevating device includes rail side plates, cuboidal blocks, track chain links, a chain, a gear, a third motor, and a U-shaped bottom plate.

The third motor drives, by means of the gear, the chain and the track chain links to rotate on the rail side plates to then drive the cuboidal blocks to move on the U-shaped bottom plate, allowing for elevation of coarse and fine aggregates.

The clay mixing device includes a mixing barrel, a transmission rod, a mixing head, a second water supply pipeline, a second motor, a discharge port, and a second cover plate.

The mixing barrel is a cylindrical barrel with an opening in the top, the second motor actuates the transmission rod and drives the mixing head to rotate to evenly blend and mix dry clay powder discharged by the discharge guide slot, the aggregates, and water sprayed by the second water supply pipeline, a finished product is discharged through the discharge port, and the second cover plate is above the discharge port.

The clay filling device includes a square guide slot, a mutually crossed and connected rod mechanism, a hydraulic jacking device, and a push plate.

The square guide slot is obliquely fixed on the model tank, and the hydraulic jacking device drives the mutually crossed and connected rod mechanism to elevate to deliver the finished product into the model tank by means of the push plate.

Further preferably, the gravel layer has a height of 0.25 m, and the sandy clay supporting layer has a height of 0.3 m; the actuator is slidably fixed onto the top cross beam of the model tank, and has a degree of freedom in two directions that are parallel to and perpendicular to the top cross beam, respectively; and the actuator can apply a dynamic load in a form of sine waves, half-sine waves or the like.

Further preferably, a side door is disposed on a side wall of the model tank, and the side door can reduce an operating height for clay filling and shorten a length of the square guide slot; a glass window is installed on a side wall opposite to the side door, facilitating observation of an internal condition of the model tank by means of the glass window.

Further preferably, the model tank device is further provided with a first water supply pipeline for wetting the model tank after the finished product is filled, to maintain the performance of the finished product.

Further preferably, the helical blade set can sufficiently crush dry clay blocks under the drive of the first motor; a mesh screen is disposed inside the clay crushing device to prevent incompletely crushed dry clay blocks from entering the mixing barrel via the discharge guide slot; and completely crushed raw materials fall into the mixing barrel through the discharge guide slot, and a rotation speed of the first motor is adjustable to thus control a discharge speed.

Further preferably, the annular second water supply pipeline is fixed inside the mixing barrel, the second water supply pipeline is connected to an external flow meter and an external water pressure booster pump, and a total volume of water entering the mixing barrel is controlled by means of the water pressure booster pump and the flow meter, allowing for configuration of a soft clay foundation with a specific water content; and the second water supply pipeline is annular and fixed to an inner wall of the mixing barrel, facilitating more uniform distribution of water in a soft clay mix.

Further preferably, the third motor drives the gear to rotate, the gear drives, by means of meshing, the chain to rotate, and each section of the chain is correspondingly and fixedly connected to each section of the track chain links, allowing the track chain links to rotate along with rotation of the chain; and the cuboidal blocks are fixed on some of the track chain links, dividing a space inside the rounded-rectangular rail side plates into compartments one after another and allowing for circulating rotation in the round-rectangular rail.

Further preferably, the bottom plate above the mixing barrel is removed to form a hole, and the coarse-fine aggregates fall into the mixing barrel when moving to the hole of the U-shaped bottom plate.

Further preferably, a bottom of the square guide slot directly faces the discharge port, a top of the square guide slot is communicated to an interior of the model tank, such that the finished product is pushed into the model tank; two hydraulic jacking devices are provided in the square guide slot to jack the mutually crossed and connected rod mechanism based on a parallelogram principle, thereby pushing the finished products attached onto a bottom plate of the square guide slot into the model tank; and during discharging from the discharge port, the mutually crossed and connected rod mechanism is retracted, and the finished products fall onto the push plate by means of the discharge port.

A method for automated configuration of a soft clay foundation based on a model tank test includes the steps of:

Step 1, turning on the first motor to allow the first motor to operate, opening the first cover plate, then, putting dry clay blocks into the clay crushing chamber via the feed port guard plate, crushing the dry clay blocks under the action of the helical blade set, passing acceptable clay powder through the discharge guide slot into the mixing barrel, and turning off the first motor after a clay crushing procedure is completed;

Step 2, closing the first cover plate while ensuring the second cover plate is closed, turning on the third motor, putting a desired mass of aggregates into the compartments divided by the cuboidal blocks on the bottom of the U-shaped bottom plate, allowing the aggregates to reach a top end of the U-shaped bottom plate as the third motor rotates to thus put the aggregates into the mixing barrel, and turning off the third motor after aggregate delivery is completed;

Step 3, turning on the second motor to simultaneously convey a preset mass of water into the mixing barrel by means of the second water supply pipeline, and turning off the second motor when an observed mix state meets a test requirement; and

Step 4, making sure the mutually crossed and connected rod mechanism is retracted, opening the second cover plate, turning on the second motor again, delivering all the finished products into the square guide slot, turning on the hydraulic jacking device, and delivering the completely mixed finished product into the model tank.

The invention has the following advantageous effects:

First, compared with the test in the prior art, the model tank scale test of the invention has the advantages of low cost, relatively reliable test results, easy operation, diverse simulated working conditions and the like. In addition, in most of the related model tank tests, the configuration and filling of the soft clay foundation is a cumbersome and time-consuming process. The invention effectively shortens the time of this process without affecting the overall test effect, and improves the function and working efficiency of the model tank, achieving more satisfactory test results.

Second, the actuator has the degree of freedom in both the parallel direction and the perpendicular direction, and can move within a certain range in the direction parallel to the cross beam to allow for loading at different positions in the horizontal direction of the road embankment surface; and meanwhile, the actuator can also move in the direction perpendicular to the horizontal plane, so as to adapt to different road embankment heights under different working conditions.

Third, the cuboidal blocks in the aggregate elevating device divide the rounded-rectangular rail into the compartments one after another; the cuboidal blocks move at a certain speed in the rounded-rectangular rail under the drive the third motor, thereby delivering raw aggregates at a lower place into the mixing barrel by means of such elevation; and the chain and track chain links in circulating rotation ensure the continuous operation of the aggregate elevating device and ensure the normal operation of this procedure, which greatly improves the aggregate delivery efficiency.

Fourth, the scissor type elevating device disposed in the clay filling device can produce a great pushing force under the action of the hydraulic jacking device, such that the finished products can be effectively pushed into the model tank. The mutually crossed rod mechanism is retracted when the clay mixing device performs discharging at the discharge port; after discharging is performed to a certain extent, the mutually crossed rod mechanism is jacked open under the action of the hydraulic jacking device according to the parallelogram principle; under the jacking force provided by the hydraulic jacking device, the push plate applies this force to the finished products; and when the finished products have a higher water content and a larger adhesive force, this jacking force is greater than the bonding force between the finished products and the bottom plate of the square guide slot, such that the finished products move upwards diagonally in the direction of the guide slot. This design can effectively prevent the finished products with higher water content from remaining on the square guide slot.

### DESCRIPTION OF THE DRAWINGS

FIG. 1 is a schematic diagram of an overall structure of a device according to the invention.

FIG. 2 is a schematic structural diagram of a clay crushing device according to the invention.

FIG. 3 is a schematic diagram of an inner structure of a clay crushing chamber according to the invention.

FIG. 4 is a schematic diagram of a coarse-fine aggregate elevating device according to the invention.

FIG. 5 is an enlarged view of the top of the coarse-fine aggregate elevating device according to the invention.

FIG. 6 is a schematic structural diagram of a clay mixing device according to the invention.

FIG. 7 is a schematic structural diagram of a clay filling device according to the invention.

In the drawings, reference signs are as follows: 10, model tank; 11, gravel layer; 12, sandy clay supporting layer; 13, rigid end-bearing pile; 14, actuator; 15, first water supply pipeline; 16, top cross beam; 17, side door; 20, feed port guard plate; 21, clay crushing chamber; 22, helical blade set; 23, mesh screen; 24, first motor; 25, discharge guide slot; 26, first cover plate; 27, belt; 28, guard plate; 30, mixing barrel; 31, transmission rod; 32, mixing head; 33, second water supply pipeline; 34, second motor; 35, flow meter; 36, water pressure booster pump; 37, discharge port; 38, second cover plate; 40, rail side plate; 41, cuboidal block; 42, track chain link; 43, chain; 44, gear; 45, third motor; 46, U-shaped bottom plate; 50, square guide slot; 51, rod mechanism; 52, hydraulic jacking device; 53, push plate.

### DETAILED DESCRIPTION OF THE EMBODIMENTS

In the description of the invention, it should be understood that the terms such as “left”, “right”, “upper”, and “lower” indicate directions or positional relations based on the directions or positional relations as shown in the accompanying drawings only for the convenience of describing the invention and simplifying the description, instead of indicating or implying that a mentioned device or element must have a specific direction or must be constructed and operated in a specific direction. The terms “first” and “second” do not indicate the level. Therefore, these terms should not be construed as limiting the invention. The specific dimensions used in the embodiments are intended only to illustrate the technical solutions, rather than limiting the protection scope of the invention.

The invention will be further described in details below in conjunction with the accompanying drawings and specific preferred embodiments.

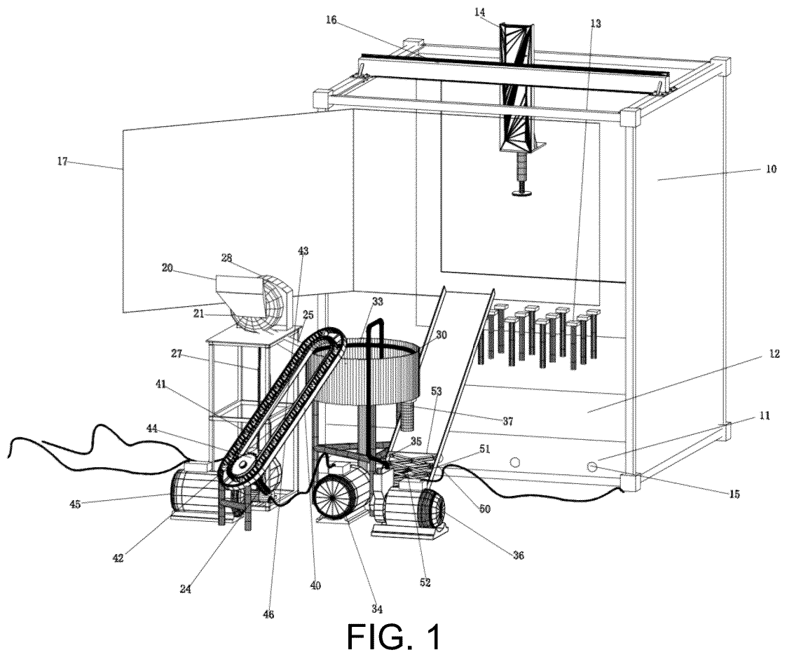

As shown in FIG. 1, a device for automated configuration of a soft clay foundation based on a model tank test includes: a model tank device, a clay crushing device, a clay mixing device, a coarse-fine aggregate elevating device, and a clay filling device.

The model tank device includes a model tank 10, a gravel layer 11, a sandy clay supporting layer 12, rigid end-bearing piles 13, an actuator 14, a first water supply pipeline 15, and a top cross beam 16.

The model tank 10 is a square tank with an opening in the top, and the gravel layer 11 is laid on the bottom of the model tank 10, the sandy clay supporting layer 12 is laid above the gravel layer, and a plurality of rigid end-bearing piles 13 is embedded in the sandy clay supporting layer 12. The top cross beam 16 is fixed to the top of the model tank 10, and the actuator 14 is suspended on the top cross beam 16.

A side door 17 is disposed on a side wall of the model tank 10, and the side door 17 can reduce an operating height for clay filling and shorten a length of the square guide slot 50; a glass window is installed on a side wall opposite to the side door 17, facilitating observation of an internal condition of the model tank 10 by means of the glass window.

The model tank device is further provided with a first water supply pipeline 15 for wetting the model tank 10 after the finished product is filled, to maintain the performance of the finished product.

The height of the gravel layer 11 is 0-0.25 m, and the height of the sandy clay supporting layer 12 is 0-0.3 m. The actuator 14 is slidably fixed onto the top cross beam 16 of the model tank 10, can be adjusted in position by means of a bolt hole in the cross beam, and has a degree of freedom in two directions that are parallel to and perpendicular to the top cross beam 16, respectively. The actuator may move within a certain range in the direction parallel to the cross beam to allow for loading at different positions in the horizontal direction on the road embankment surface; and meanwhile, the actuator may also move in the direction perpendicular to the horizontal surface to adapt to different road embankment heights under different working conditions. The actuator 14 may apply a dynamic load in a form of sine waves, half-sine waves or the like.

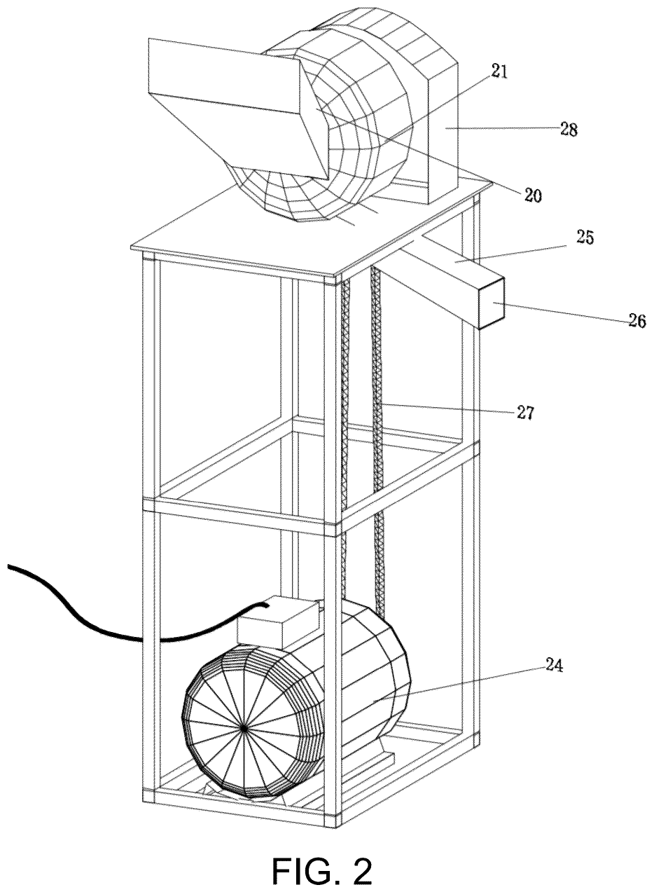

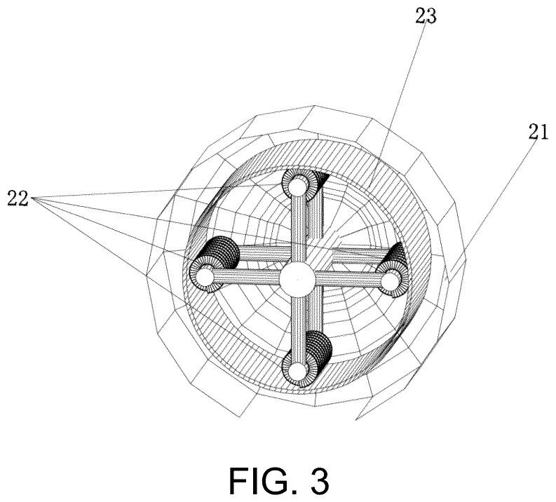

As shown in FIG. 2 and FIG. 3, the clay crushing device includes a feed port guard plate 20, a clay crushing chamber 21, a helical blade set 22, a first motor 24, a discharge guide slot 25, a first cover plate 26, and a belt 27.

The feed port guard plate 20 is obliquely and fixedly connected to the clay crushing chamber 21, the helical blade set 22 is disposed inside the clay crushing chamber 21, a lower part of the clay crushing chamber 21 is connected to the discharge guide slot 25, and the first motor 24 drives, by means of the belt 27, the helical blade set 22 to rotate. The first cover plate 26 is located at an outlet of the discharge guide slot 25, which is closed when discharging is not performed. A guard plate 28 covers the belt 27 to prevent injuries caused by a rotating machine.

The helical blade set 22 can sufficiently crush dry clay blocks under the drive of the first motor 24. A mesh screen 23 is disposed inside the clay crushing device to prevent incompletely crushed dry clay blocks from entering the mixing barrel 30 via the discharge guide slot 25. Completely crushed raw materials fall into the mixing barrel 30 through the discharge guide slot 25, and the rotation speed of the first motor 24 is adjustable to thus control a discharge speed. Four helical blade sets rotating coaxially are disposed inside the clay crushing device, which can greatly improve the clay crushing efficiency for larger clay blocks. Meanwhile, the mesh screen distributed around the helical blade sets can achieve a filtering effect to prevent the incompletely crushed clay blocks from entering the mixing barrel.

The first cover plate is disposed at the outlet of the discharge guide slot of the clay crushing device, and can be opened or closed according to test needs; and the second cover plate is above the discharge port of the clay mixing device, and can be opened or closed according to test procedure needs.

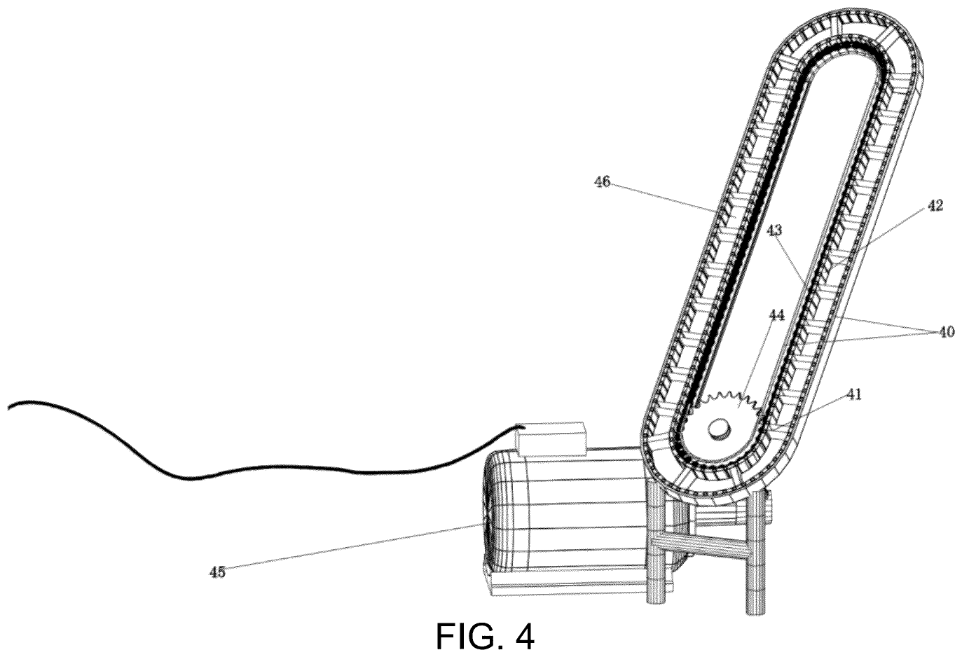

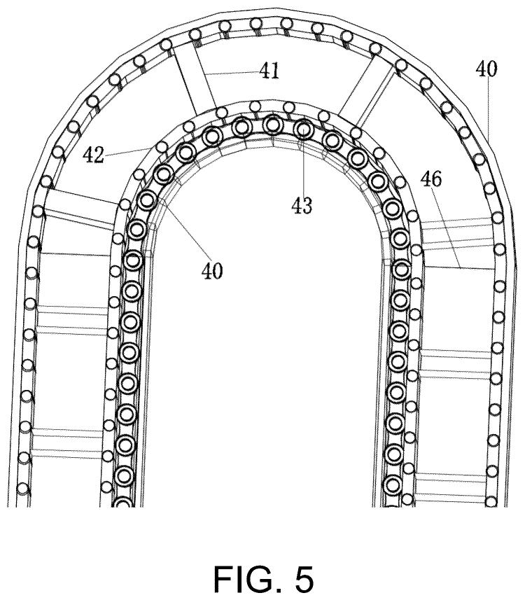

As shown in FIG. 4 and FIG. 5, the coarse-fine aggregate elevating device includes rail side plates 40, cuboidal blocks 41, track chain links 42, a chain 43, a gear 44, a third motor 45, and a U-shaped bottom plate 46.

The third motor 45 drives the gear 44 to rotate, the gear 44 drives, by means of meshing, the chain 43 to rotate, and each section of the chain 43 is correspondingly and fixedly connected to each section of the track chain links 42, allowing the track chain links 42 to rotate along with rotation of the chain 43. The cuboidal blocks 41 are fixed on some of the track chain links 42, dividing a space inside the rounded-rectangular rail side plates 40 into compartments one after another and allowing for circulating rotation in the round-rectangular rail.

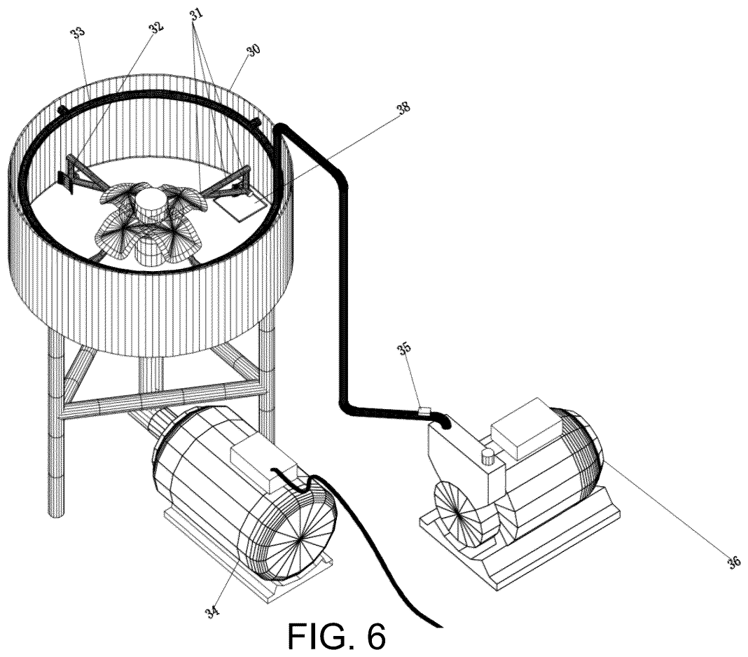

As shown in FIG. 6, the clay mixing device includes a mixing barrel 30, a transmission rod 31, a mixing head 32, a second water supply pipeline 33, a second motor 34, a discharge port 37, and a second cover plate 38.

The mixing barrel 30 is a cylindrical barrel with an opening in the top, the second motor 34 actuates the transmission rod 31 and drives the mixing head 32 to rotate to evenly blend and mix dry clay powder discharged by the discharge guide slot 25, the aggregates, and water sprayed by the second water supply pipeline 33, and a finished product is discharged through the discharge port 37. The bottom plate above the mixing barrel 30 is removed to form a hole, and the coarse-fine aggregates fall into the mixing barrel 30 when moving to the hole of the U-shaped bottom plate 46. The second cover plate 38 is above the discharge port 37 and is configured to close the discharge port when discharging is not needed.

The transmission rod in the mixing barrel consists of three rods, among which a stable triangular structure is formed. Such a structure is conducive to improve the stability of the clay mixing device, and also shows a significant effect of enhancing the mixing of the clay mixing system.

The annular second water supply pipeline 33 is fixed inside the mixing barrel 30, the second water supply pipeline 33 is connected to an external flow meter 35 and an external water pressure booster pump 36, and a total volume of water entering the mixing barrel 30 is controlled by means of the water pressure booster pump 36 and the flow meter 35, allowing for configuration of a soft clay foundation with a specific water content. The second water supply pipeline 33 is annular and fixed to an inner wall of the mixing barrel 30, facilitating more uniform distribution of water in a soft clay mix.

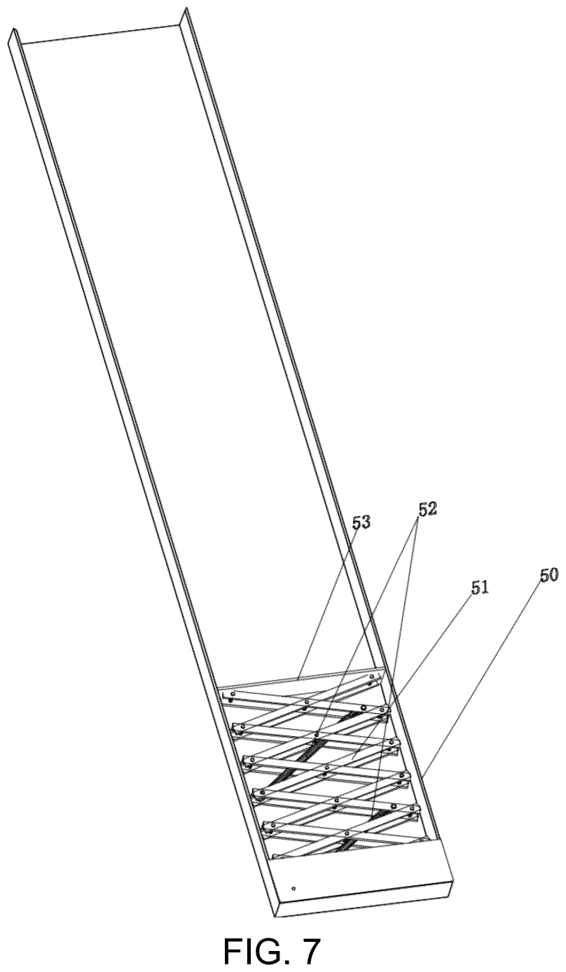

As shown in FIG. 7, the clay filling device includes a square guide slot 50, a mutually crossed and connected rod mechanism 51, a hydraulic jacking device 52, and a push plate 53.

The bottom of the square guide slot 50 directly faces the discharge port 37, the top of the square guide slot is communicated to an interior of the model tank 10, such that the finished product is pushed into the model tank 10. Two hydraulic jacking devices 52 are provided in the square guide slot 50, and the hydraulic jacking devices are scissor type elevating devices. The mutually crossed and connected rod mechanism 51 is jacked according to the parallelogram principle, thereby pushing the finished products attached onto a bottom plate of the square guide slot 50 into the model tank 10. During discharging from the discharge port 37, the mutually crossed and connected rod mechanism 51 is retracted, and the finished products fall onto the push plate 53 by means of the discharge port 37.

Based on the device mentioned above, there is provided a method for automated configuration of a soft clay foundation based on a model tank test, which effectively shortens the time of procedures without affecting the overall test effect, and improves the function and working efficiency of the model tank, achieving more satisfactory test results.

The method specifically includes the steps of:

Step 1, turning on the first motor 24 to allow the first motor to operate, opening the first cover plate 26, then, putting dry clay blocks into the clay crushing chamber 21 via the feed port guard plate 20, crushing the dry clay blocks under the action of the helical blade set 22, passing acceptable clay powder through the discharge guide slot 25 into the mixing barrel 30, and turning off the first motor 24 after a clay crushing procedure is completed;

Step 2, closing the first cover plate 26 while ensuring the second cover plate 38 is closed, turning on the third motor 45, putting a desired mass of aggregates into the compartments divided by the cuboidal blocks 41 on the bottom of the U-shaped bottom plate 46, allowing the aggregates to reach a top end of the U-shaped bottom plate 46 as the third motor 43 rotates to thus put the aggregates into the mixing barrel 30, and turning off the third motor 45 after aggregate delivery is completed;

Step 3, turning on the second motor 34 to simultaneously convey a preset mass of water into the mixing barrel 30 by means of the second water supply pipeline 33, and turning off the second motor 34 when an observed mix state meets a test requirement; and

Step 4, making sure the mutually crossed and connected rod mechanism 51 is retracted, opening the second cover plate 38, turning on the second motor 34 again, delivering all the finished products into the square guide slot 50, turning on the hydraulic jacking device 52, and delivering the completely mixed finished product into the model tank 10.

The detailed description above provides the preferred embodiments of the invention. However, the invention is not limited to the details in the above embodiments. A variety of equivalent transformations can be made to the technical solutions of the invention within the technical conception scope of the invention, and these equivalent transformations shall fall within the protection scope of the invention.

1. A device for automated configuration of a soft clay foundation based on a model tank test, wherein comprising a model tank device, a clay crushing device, a clay mixing device, a coarse-fine aggregate elevating device and a clay filling device,
wherein the model tank device comprises a model tank, a gravel layer, a sandy clay supporting layer, rigid end-bearing piles, an actuator, and a top cross beam;
the model tank is a square tank with an opening in a top, and the gravel layer is laid on a bottom of the model tank, the sandy clay supporting layer is laid above the gravel layer, and a plurality of rigid end-bearing piles is embedded in the sandy clay supporting layer; the top cross beam is fixed to the top of the model tank, and the actuator is suspended on the top cross beam;
the clay crushing device comprises a feed port guard plate, a clay crushing chamber, a helical blade set, a first motor, a discharge guide slot, a first cover plate, and a belt;
the feed port guard plate is obliquely and fixedly connected to the clay crushing chamber, the helical blade set is disposed inside the clay crushing chamber, a lower part of the clay crushing chamber is connected to the discharge guide slot, and the first motor drives, by means of the belt, the helical blade set to rotate; the first cover plate is located at an outlet of the discharge guide slot;
the coarse-fine aggregate elevating device comprises rail side plates, cuboidal blocks, track chain links, a chain, a gear, a third motor, and a U-shaped bottom plate;
the third motor drives, by means of the gear, the chain and the track chain links to rotate on the rail side plates to then drive the cuboidal blocks to move on the U-shaped bottom plate, allowing for elevation of coarse and fine aggregates;
the clay mixing device comprises a mixing barrel, a transmission rod, a mixing head, a second water supply pipeline, a second motor, a discharge port, and a second cover plate;
the mixing barrel is a cylindrical barrel with an opening in a top, the second motor actuates the transmission rod and drives the mixing head to rotate to evenly blend and mix dry clay powder discharged by the discharge guide slot, the aggregates, and water sprayed by the second water supply pipeline, a finished product is discharged through the discharge port, and the second cover plate is above the discharge port;
the clay filling device comprises a square guide slot, a mutually crossed and connected rod mechanism, at least one hydraulic jacking device, and a push plate; and
the square guide slot is obliquely fixed on the model tank, the hydraulic jacking device drives the mutually crossed and connected rod mechanism to elevate to deliver a finished product into the model tank by means of the push plate.

2. The device for automated configuration of the soft clay foundation based on the model tank test according to claim 1, wherein the gravel layer has a height of 0.25 m, and the sandy clay supporting layer has a height of 0.3 m; the actuator is slidably fixed onto the top cross beam of the model tank, and has a degree of freedom in two directions that are parallel to and perpendicular to the top cross beam, respectively; the actuator can apply a dynamic load in a form of sine waves, half-sine waves or the like.

3. The device for automated configuration of the soft clay foundation based on the model tank test according to claim 1, wherein a side door is disposed on a side wall of the model tank, and the side door can reduce an operating height for clay filling and shorten a length of the square guide slot; a glass window is installed on a side wall opposite to the side door, facilitating observation of an internal condition of the model tank by means of the glass window.

4. The device for automated configuration of the soft clay foundation based on the model tank test according to claim 1, wherein the model tank device is further provided with a first water supply pipeline for wetting the model tank after the finished product is filled, to maintain a performance of the finished product.

5. The device for automated configuration of the soft clay foundation based on the model tank test according to claim 1, wherein the helical blade set can sufficiently crush dry clay blocks under the drive of the first motor; a mesh screen is disposed inside the clay crushing device to prevent incompletely crushed dry clay blocks from entering the mixing barrel via the discharge guide slot; and completely crushed raw materials fall into the mixing barrel through the discharge guide slot, and a rotation speed of the first motor is adjustable to thus control a discharge speed.

6. The device for automated configuration of the soft clay foundation based on the model tank test according to claim 1, wherein the annular second water supply pipeline is fixed inside the mixing barrel, the second water supply pipeline is connected to an external flow meter and an external water pressure booster pump, and a total volume of water entering the mixing barrel is controlled by means of the water pressure booster pump and the flow meter, allowing for configuration of the soft clay foundation with a specific water content; and the second water supply pipeline is annular and fixed to an inner wall of the mixing barrel, facilitating more uniform distribution of water in a soft clay mix.

7. The device for automated configuration of the soft clay foundation based on the model tank test according to claim 1, wherein the third motor drives the gear to rotate, the gear drives, by means of meshing, the chain to rotate, and each section of the chain is correspondingly and fixedly connected to each section of the track chain links, allowing the track chain links to rotate along with rotation of the chain; and the cuboidal blocks are fixed on some of the track chain links, dividing a space inside a rounded-rectangular rail side plates into compartments one after another and allowing for circulating rotation in a round-rectangular rail.

8. The device for automated configuration of the soft clay foundation based on the model tank test according to claim 1, wherein a bottom plate above the mixing barrel is removed to form a hole, and coarse-fine aggregates fall into the mixing barrel when moving to the hole of the U-shaped bottom plate.

9. The device for automated configuration of the soft clay foundation based on the model tank test according to claim 1, wherein a bottom of the square guide slot directly faces the discharge port, a top of the square guide slot is communicated to an interior of the model tank, such that the finished product is pushed into the model tank; two hydraulic jacking devices are provided in the square guide slot to jack the mutually crossed and connected rod mechanism based on a parallelogram principle, thereby pushing the finished products attached onto a bottom plate of the square guide slot into the model tank; and during discharging from the discharge port, the mutually crossed and connected rod mechanism is retracted, and the finished products fall onto the push plate by means of the discharge port.

10. A method for automated configuration of the soft clay foundation based on the model tank test according to claim 1, wherein comprising the steps of:
Step 1, turning on the first motor to allow the first motor to operate, opening the first cover plate, then, putting dry clay blocks into the clay crushing chamber via the feed port guard plate, crushing the dry clay blocks under an action of the helical blade set, passing acceptable clay powder through the discharge guide slot into the mixing barrel, and turning off the first motor after a clay crushing procedure is completed;
Step 2, closing the first cover plate while ensuring the second cover plate is closed, turning on the third motor, putting a desired mass of aggregates into compartments divided by the cuboidal blocks on a bottom of the U-shaped bottom plate, allowing the aggregates to reach a top end of the U-shaped bottom plate as the third motor rotates to thus put the aggregates into the mixing barrel, and turning off the third motor after aggregate delivery is completed;
Step 3, turning on the second motor to simultaneously convey a preset mass of water into the mixing barrel by means of the second water supply pipeline, and turning off the second motor when an observed mix state meets a test requirement; and
Step 4, making sure the mutually crossed and connected rod mechanism is retracted, opening the second cover plate, turning on the second motor again, delivering all the finished products into the square guide slot, turning on the hydraulic jacking device, and delivering a completely mixed finished product into the model tank.
